(19) United States
(12) Patent Application Publication
(10) Pub. No.: US 2004/0131558 A1
(43) Pub. Date: Jul. 8, 2004

(54) Oral disease prevention and treatment

(76) Inventor: Douglas J. Hauck, Beverly Hills, CA (US)

Correspondence Address: Douglas J. Hauck, DDS, Suite 625, 8920 Wilshire Boulevard, Beverly Hills, CA, 90211, US

(21) Appl. No.: 10/601,137

(22) Filed: Jun. 20, 2003

Related U.S. Application Data
(60) Provisional application No. 60/390,089, filed on Jun. 21, 2002.
(60) Provisional application No. 60/423,605, filed on Nov. 4, 2002.

(51) Int. Cl. A61K 7/16; A61C 5/00

(52) U.S. Cl. 424/49; 433/217.1

(57) ABSTRACT

One aspect of the invention provides a convenient method for delivering a composition orally to a patient. The method utilizes teeth, either natural or artificial, including implants and dentures, and/or any modification to the teeth, such as, but not limited to, crowns, inlays, onlays and fillings. In some instances, such as with natural or artificial teeth, a void is made in the substrate, and the composition containing the desired chemical, preferably in a biodegradable, biocompatible gel, is placed in the void, where it is gradually released. In a variation, the composition is incorporated into a filling, a root canal treatment, the glaze of a crown or artificial tooth, or the plastic base of dentures.

### FIELD OF THE INVENTION

The present invention is in the field of oral hygiene, including prevention and treatment of unwanted conditions in the oral cavity. It is also in the field of drug delivery.

### BACKGROUND

Oral hygiene is an important part of health maintenance. This is especially true when it comes to abnormal and/or undesirable conditions of the mouth, some of which are caused by excess growth of detrimental bacteria in the oral cavity. Such growth can contribute to a variety of conditions, including malodorous breath (also called halitosis, or bad breath), tooth caries, and gum disease.

Many attempts have been made to solve the problem of malodorous breath, as it occurs in many people, and affects social interactions. It has been estimated that 20-90 million people in the United States alone suffer from this condition. This widespread condition has spawned a large industry that has attempted to provide its solution. Products in this area range from breath mints and lozenges to mouthwashes, sprays, dissolvable films, gums, coated strips and other oral appliances. Most recently, the use of tongue scraping has been promoted as a method of removing the unwanted sources of the offending odors.

While the industry has been busy, the products have been less than successful. Use of some of these products can cause socially unacceptable activity such as sucking and chewing. Use of others can leave a sticky feeling or bad taste. Use of the oral appliances is very invasive and can interfere with speech. Further, such products only provide temporary masking of the odor.

One option for treatment is the repeated administration of compositions to the patient. However, this requires repeated visits to the dentist or other medical practitioner for stepwise incorporation of the required composition. Patient compliance in such schemes is usually very poor, and treatments are usually not completed. Thus, there is a need for a convenient, long-acting, pleasant method for treating bad breath.

Additionally, many medications, in particular those for treating conditions in the oral cavity, can be delivered orally. Studies have shown that the continuous application of chemicals can minimize the required dose, while eliminating the need for repeated visits to the dentist or other medical practitioner. However, it has been difficult to provide such medications steadily over a period of time without interfering with the patient's speech, eating and drinking.

### SUMMARY OF THE INVENTION

One aspect of the invention provides a convenient method for delivering a composition orally to a patient. The method utilizes teeth, either natural or artificial, including implants and dentures, and/or any modification to the teeth, such as, but not limited to, crowns, inlays, onlays and fillings. In some instances, such as with natural or artificial teeth, a void is made in the substrate, and the composition containing the desired chemical, preferably in a biodegradable, biocompatible gel, is placed in the void, where it is gradually released. In a variation of this method, the composition is not biodegradable, and the desired chemical is gradually released through mastication. In yet another variation, the chemical is incorporated into the substrate, such as dentures, inlays or fillings, and is released gradually into the oral cavity by mastication or the action of saliva.

In another aspect of the invention, a method is provided to prevent or treat malodorous breath using flavored fillings. A composition is delivered to the patient by any of the methods described above, such as in flavored fillings or flavored dentures. The composition is released over time at a rate sufficient to effect better breath but not at a rate high enough to affect the sense of taste of the average patient.

In yet another embodiment of the invention, a composition is delivered over time, by any of the methods described above, within the oral cavity wherein the composition is an antibacterial that lowers or eliminates the presence of undesirable bacteria in the oral cavity. The composition prevents or treats various conditions caused by the unwanted bacteria, such as malodorous breath, tooth caries and gum disease.

In another embodiment of the invention, a method of delivery of a composition for enhancing the health of the oral cavity is provided by any of the methods described above. The composition is released slowly into the oral cavity by mastication or the action of saliva. After its effective use, the composition may decay, become inactive and/or be removed.

In another embodiment of the invention, a method of delivery of a composition for treating an oral cancer is provided as described above. Radioactive seeds are placed in a void proximal to and directed at the cancerous tissue. Alternatively, a chemotherapeutic is supplied using any of the methods described above. The composition is released slowly into the oral cavity by mastication or the action of saliva. After its effective use, the composition may be removed and replaced if required.

Further, a composition for enhancing the health of a mammal by delivery in the oral cavity is provided. The desired composition is delivered by any of the methods described above. The composition is released slowly over time into the mouth and then into the gastrointestinal tract by the action of saliva.

Additionally, any of these compositions can be combined with others, unless medically indicated otherwise, to provide a multipurpose oral delivery system. Likewise, this system can be used in conjunction with other treatments or compositions in the oral cavity.

### DESCRIPTION OF THE FIGURES

FIG. 1 shows the embodiment wherein the composition is incorporated into the glazed layer (10) of a crown (12).

FIG. 2 shows the embodiment wherein a capsule (14) is placed in a void on the lingual side of a crown, wherein the capsule is refillable.

FIG. 3 shows an example of six units of veneer crowns (18) and bridge restoratives (20) on the lingual surfaces.

FIG. 4 shows an example of the composition placed in fillings (24) on different aspects of teeth (26).

FIG. 5 shows possible placements (26) for a composition in the base of upper (28) and/or lower (30) dentures.

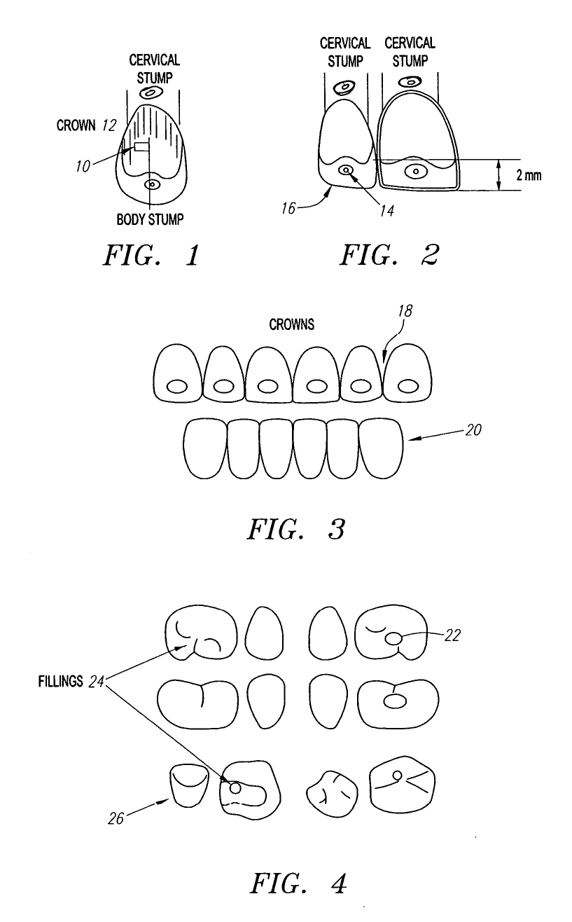

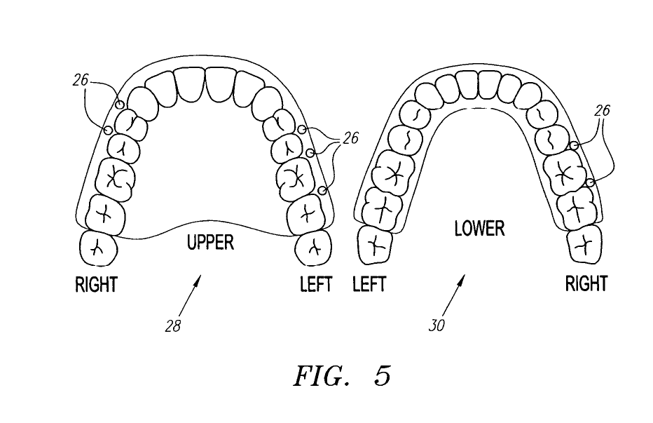

### DETAILED DESCRIPTION OF THE INVENTION

In one aspect of the invention, compositions are incorporated in, on or into teeth, modifications of teeth, or dental appliances. As used herein, the terms “tooth” and “teeth” refer to teeth (singular or plural), either natural or artificial, including but not limited to dentures, retainers, implants. “Tooth modifications” refer to such changes as crowns, fillings, inlays, onlays, veneers, root canal therapies and the like. The terms “slow release”, “time release”, “delivered over time” and “released over time” are interchangeable herein and refer to the release of the desired composition into the oral cavity over a period of time, extending anywhere from about 2 hours, about 4 hours, about 6 hours, about 8 hours, about 10 hours, about 12 hours, about 24 hours, about 36 hours, about 48 hours or about 72 hours to about half a week, about one week, or multiple weeks, or longer, after implantation in the oral cavity.

### Compositions

Any composition that is useful for delivery via the oral cavity can be used. Such compositions include, but are not limited to, flavorings such as flavored oils; anti-bacterial compositions such as penicillins and tetracyclines; odor reducers such as chlorine dioxide, zinc gluconate, other biocompatible zinc salts, and glutaraldehyde; chemotherapeutics such as doxorubicin and vincristine; radiotherapeutics such as radioactive seeds of ¹²⁵I, ¹⁹²I, palladium, and iridium; compositions to improve oral health such as sodium fluoride and potassium nitrate; anesthetics such as Lidocaine; antiseptics such as various forms of iodine; antimicrobials such as chlorhexidine; antifungals such as clotrimazole and Nystatin; anti-inflammatory agents such as ibuprofen and salicylates; antivirals; and combinations of any of the above. In particular, flavorings incorporated in fillings and/or dentures, i.e. flavored fillings and flavored dentures, are provided.

### Formulations

The compositions of this invention are released in the oral cavity over a period of time. The release allows the compositions to spread through the oral cavity, thereby potentially affecting more than the tissue immediately surrounding the composition. This slow release can be facilitated by a variety of mechanisms. In one embodiment, the composition is provided in a slow release formulation, for example biodegradable polymers, to be placed in a void in porcelain or composite in a tooth or plastic in a denture. An example of such a polymer is a water soluble hydroxyalkyl-methyl gel. Other formulations include photopolymerized biogels, such as modified polyethylene glycol, wherein the porosity and durability of the gels can be adjusted for the desired rate of release of the compound. In another embodiment, the composition is incorporated into a resin or plastic to be used to make an inlay or filling for a tooth, or to create a denture.

### Methods of Deposit

The incorporation can be performed by a variety of methods. One method involves first creating a void in the substrate. The void is preferably designed into the lingual or buccal side of the tooth or tooth modification, depending on where the chemical is to be delivered. In particular, when the composition or chemical has a flavor, it may be desirable to place the void on the buccal side, away from the tongue, so as not to interfere with taste.

The void is then filled, partly filled, or coated, with the desired composition. The chemical will be released gradually, depending on a variety of factors, including but not limited to the formulation, the amount of the composition, and the size of the opening of the void. The void can be completely enclosed by the substrate, or it can be connected with the exterior of the substrate. This procedure is especially useful with natural or artificial teeth such as implants, and with crowns.

In some instances, the void occurs naturally in the tooth or tooth modification, and need not be created. This type of void can be modified as needed for containing the composition.

For dentures, the composition can be incorporated into the plastic base, or placed in a void in one or more of the teeth.

Alternatively, the composition can be incorporated into dental bonding composite resins which can then be used as a composite filling, temporary filling, temporary crown, root canal therapy, or the like. The composition is then released slowly from the tooth modification.

In another embodiment, the composition can be placed on the tooth by glazing. For example, a crown can be formed for a patient wherein the composition is incorporated into the glazed layer around the body stump of the crown. The composition is infused into the final glaze. The combination is low-fire glazed to the exposed portion of the crown, temporary crown, inlay, onlay or veneer. The exposed portion of the tooth is preferably pre-textured for better attachment of the glaze. The pre-texturing can be performed, for example, by sandblasting the surface, using standard dental techniques.

In yet another embodiment, a flavoring agent can be placed in a bleach tray and mixed with the bleaching material in order to make use of the tray, such as overnight, more palatable to the wearer.

### Methods of Treatment

Unacceptable mouth odors can easily be treated by these methods. Odor reducing agents, singly or in combination, can be used either directly in a slow release gel, or be combined with other compounds into compositions and placed in teeth or tooth modifications. These agents, such as penicillins and tetracyclines, may be chosen to reduce or deplete the unwanted oral bacteria, thus reducing or eliminating the odor caused by the presence of the bacteria. Alternatively, the agents may be chosen for their ability to mask the temporary malodorous breath. Examples are chlorine dioxide, zinc gluconate, and glutaraldehyde. The effect is long lasting, as the agents are present in the oral cavity for a much longer time than when delivered by such items as gums and mouth washes. The agents are preferably positioned within the oral cavity at one or more locations away from the taste buds, so as not to interfere with the normal taste of food products.

Compositions such as anti-neoplastic chemotherapy can be incorporated into teeth, or tooth modifications, for the treatment of oral cancers, such as tongue and palate cancers, and pre-cancerous conditions. For example doxorubicin and/or vincristine can be used in appropriate doses, easily determinable by one skilled in the art, to treat oral cancers. The chemotherapeutic can be placed in one or more teeth or tooth modifications that are proximal to the cancerous or pre-cancerous tissue so as to minimize its effect on the remaining oral tissue. The chemotherapeutic can be combined with other compounds, as medically directed, and/or with a flavoring.

Likewise, radiation can be applied to the oral cavity using these methods. The source of the radiation can be incorporated into the composition to be placed in the mouth. In an example of this embodiment, the source can be a relatively low emitting radioisotope, for example ¹²⁵I seeds, eliminating the need for a larger external dose of the radiation, due to the ability to maintain the presence of the source at or near the desired treatment site for a period of time. Delivery is preferably by creating a crown, or using an existing crown on a tooth proximal to a cancer, with a void facing in the direction of the area to be treated. The opening of the void is preferably small so as to focus towards the treatment site. Radiation seeds are deposited in an appropriate composition into the void, and the radiation is emitted in a focused manner at the treatment site. The void is surrounded by a metal partition within the tooth to protect the remainder of the tissue from exposure to the radiation. At various time points, depending on the half-life of the radioactive chemical, the old seeds can be removed and potentially replaced by additional seeds if needed.

This same procedure can be accomplished by using dentures instead of one or more crowns. One or more voids can be made in the base and/or teeth of the dentures, wherein the voids are located proximal to cancerous tissue, and the proximal end of each void points at the cancerous tissue. As with the crown, a void in a denture is comprises a metal shield to protect the surrounding tissue. The radiation is supplied, and is replaceable, as in the use of a crown.

Another application involves the delivery of an anesthetic to the oral cavity. The anesthetic is incorporated into the composition to be placed in the mouth, either in a void or as a glaze. It can be delivered alone or in combination by any of the methods described above.

### Methods of Prevention

Additionally, various compositions can be provided in the oral cavity to prevent the occurrence of undesirable or unhealthy conditions. For example, one or more anti-bacterial agents can be provided, using any of the procedures described above, to prevent the build-up of odor or caries causing bacteria.

### Flavorings

Flavorings can be supplied in the oral cavity, for example to mask undesired tastes and/or odors. These flavorings can be provided using any of the methods described herein, by mixing the flavoring compounds into the compositions. The flavorings can be supplied alone or in combination with other compounds. Preferably, the flavorings do not interfere with the normal taste of food products. In a preferred embodiment, the one or more flavorings are located on the buccal side of the teeth to keep them away from the tongue.

### EXAMPLES

### Example 1

Reduction In Malodorous Breath

A composition is made as a biodegradable gel to diminish or eliminate malodorous breath. The composition comprises 10% hydroxypropyl methyl cellulose polymer, 7% corn starch, 5% flavoring, 0.3% polysorbate 80, 0.2% zinc gluconate, 0.2% Sucralose or other sweetener, 75% water, and optional colorant. The composition is thoroughly mixed and an appropriate sized portion is placed in a void in one or more teeth and/or tooth modifications in a patient's mouth. The cellulose works to extinguish the odor at its source, while the flavoring, such as mint, mixes with the patient's breath, masking the odor. The flavoring is supplied at a level low enough so as not to interfere with the patient's taste.

An odor suppressor such as chlorine dioxide, and/or odor reducer such as chlorohexidine, can be added to the composition to enhance its effectiveness. In addition to the flavoring and optional odor suppressors, any of the compositions listed above, including any combination thereof, can be included.

### Example 2

Delivery of a Medication Via a Crown

The external portion of a crown is pre-textured by sandblasting the surface, using standard dental techniques. A dose of a medication appropriate for slow release into the oral cavity infused into the final glaze and placed on the textured surface. The combination is low-fire glazed to the exposed portion of the crown. The medication is released slowly into the oral cavity, and continues to be released over a period of time, depending on the dosage incorporated in the glaze. The medication can comprise any of the compositions listed above, including any combination thereof.

### Example 3

Method for Treatment of Tongue Cancer Using Radiation

A predetermined amount of ¹²⁵I radioactive seeds is mixed in the composition of example 1. The flavoring of the composition is optional. A porcelain crown is created for a tooth that is proximal to the cancerous tissue when the tongue at rest. The crown has an underlying metal layer that acts as a shield to keep the radiation from damaging surrounding tissue. A small void is created in the crown so that the outer, open end of the void points at the cancerous tissue. The radiation composition is placed inside the void, and radiation is thereby directed at the cancerous tissue. Radiation is emitted over a period of time, depending on the amount and type of isotope being used. If further radiation is needed after depletion of the first radioactive seeds, the used composition can be removed and replaced with a fresh supply. The radioactive seeds can be supplied with any of the other compositions listed above, including combinations thereof.

### Example 4

Delivery of a Medication Via a Filling

A medication is mixed with a composite to be used in a filling. Preferably small amount of a flavoring such as mint oil is added. The mixture is then placed in the tooth using methods known to those of skill in the art. The medication, and the flavoring if used, is released during mastication when pressure is applied to the area of the filling. This results in a slow release of the medication over time. The medication can comprise any of the compositions listed above, including any combination thereof.

### Example 5

Second Method of Delivery of a Medication Via a Filling

A medication, optionally with a flavoring, is mixed with the final glaze material to cover a porcelain filling. The composition is placed over the porcelain and low-fire glazed in situ. The medication, and optionally the flavoring, is released over time from the glazing into the oral cavity. The medication can comprise any of the compositions listed above, including any combination thereof.

### Example 6

Delivery of a Medication Via Dentures

A medication is placed in a void in one or more teeth of the denture as described in Example 1. The medication can be replaced or refreshed by removing the composition such as by drilling, and applying the new composition. The medication can comprise any of the compositions listed above, including any combination thereof.

### Example 7

Second Method of Delivery of a Medication Via Dentures

A medication is mixed with the components used to form the plastic base of a set of dentures. It can be used on the upper dentures, the lower dentures, or both. The plastic is cured, thereby incorporating the medication. The medication is released from the plastic by movement of the dentures during mastication. The medication can comprise any of the compositions listed above, including any combination thereof.

I claim:

1. A method for delivery of a composition in the oral cavity comprising the steps of
a) creating, or utilizing an existing, void in one or more teeth or tooth modifications, and
b) supplying to the void the composition in a slow-release form.

2. The method of claim 1 wherein the one or more teeth are selected from the group consisting of natural teeth, implants and dentures.

3. The method of claim 1 wherein the one or more tooth modifications are selected from the group consisting of fillings, crowns, temporary crowns and root canal therapies.

4. The method of claim 3 wherein the composition is incorporated in a composite used as a filling.

5. The method of claim 3 wherein the composition is placed inside the crown.

6. The method of claim 5 wherein the crown is semi-porous.

7. The method of claim 1 wherein the composition is selected from the group consisting of flavorings, anti-bacterials, odor reducers, chemotherapeutics, radiotherapeutics, compositions to improve oral health, anesthetics, antiseptics, antimicrobials, antifungals, anti-inflammatories, antivirals, and combinations thereof.

8. The method of claim 7 wherein the flavorings are flavored oils.

9. The method of claim 7 wherein the odor reducers are selected from the group consisting of chlorine dioxide, zinc gluconate, other biocompatible zinc salts, chlorohexidine, glutaraldehyde, scents and combinations thereof.

10. The method of claim 7 wherein the chemotherapeutics are doxorubicin, vincristine, or a combination thereof.

11. The method of claim 7 wherein the radiotherapeutics are selected from the group consisting of radioactive seeds of ¹²⁵I, ¹⁹²I, palladium, iridium and combinations thereof.

12. The method of claim 1 wherein the composition is selected from the group consisting of penicillins, tetracyclines, sodium fluoride, potassium nitrate, Lidocaine, iodine containing compounds, chlorhexidine, clotrimazole, Nystatin, ibuprofen and salicylates.

13. The method of claim 1 wherein the composition is used to treat malodorous breath.

14. The method of claim 1 wherein the composition is used to decrease the amount of undesirable microbes in the oral cavity.

15. The method of claim 1 wherein the composition is used to maintain the health of the oral cavity.

16. The method of claim 1 wherein the composition is used to maintain the health of a mammalian body.

17. The method of claim 1 wherein the slow release formulation is comprised of a biodegradable, biocompatible gel.

18. The method of claim 16 wherein the gel comprises hydroalkyl methyl cellulose.

19. A method for delivery of a composition in the oral cavity comprising the steps of
a) mixing the composition in glazing material to form a glazing mixture, and
b) low firing the glazing mixture onto a tooth surface selected from the group consisting of crowns, inlays, onlays and veneers.

20. The method of claim 19 wherein the tooth surface is roughened before being placed in contact with the glazing mixture.

21. The method of claim 19 wherein the composition is selected from the group consisting of flavorings, anti-bacterials, odor reducers, chemotherapeutics, radiotherapeutics, compositions to improve oral health, anesthetics, antiseptics, antimicrobials, antifungals, anti-inflammatories, antivirals, and combinations thereof.

22. The method of claim 21 wherein the flavorings are flavored oils.

23. The method of claim 21 wherein the odor reducers are selected from the group consisting of chlorine dioxide, zinc gluconate, other biocompatible zinc salts, chlorohexidine, glutaraldehyde, scents and combinations thereof.

24. The method of claim 21 wherein the chemotherapeutics are doxorubicin, vincristine, or a combination thereof.

25. The method of claim 21 wherein the radiotherapeutics are selected from the group consisting of radioactive seeds of ¹²⁵I, ¹⁹²I, palladium, iridium and combinations thereof.

26. The method of claim 21 wherein the composition is selected from the group consisting of penicillins, tetracyclines, sodium fluoride, potassium nitrate, Lidocaine, iodine containing compounds, chlorhexidine, clotrimazole, Nystatin, ibuprofen and salicylates.

27. The method of claim 21 wherein the composition is used to treat malodorous breath.

28. The method of claim 21 wherein the composition is used to decrease the amount of undesirable microbes in the oral cavity.

29. The method of claim 21 wherein the composition is used to maintain the health of the oral cavity.

30. The method of claim 21 wherein the composition is used to maintain the health of a mammalian body.

31. A dental device for the delivery of a composition to the oral cavity comprised of an artificial tooth or tooth modification, a void in said tooth or tooth modification, and a composition within said void, wherein the composition comprises a slow release gel and a desired compound.

32. The device of claim 31 wherein the artificial tooth is an implant.

33. The device of claim 31 wherein the tooth modification is a crown.

34. The device of claim 31 wherein the compound is selected from the group consisting of flavorings, anti-bacterials, odor reducers, chemotherapeutics, radiotherapeutics, compositions to improve oral health, anesthetics, antiseptics, antimicrobials, antifungals, anti-inflammatories, antivirals, and combinations thereof.

35. The device of claim 34 wherein the flavorings are flavored oils.

36. The device of claim 34 wherein the odor reducers are selected from the group consisting of chlorine dioxide, zinc gluconate, other biocompatible zinc salts, chlorohexidine, glutaraldehyde, scents and combinations thereof.

37. The device of claim 34 wherein the chemotherapeutics are doxorubicin, vincristine, or a combination thereof.

38. The device of claim 34 wherein the radiotherapeutics are selected from the group consisting of radioactive seeds of ¹²⁵I, ¹⁹²I, palladium, iridium and combinations thereof.

39. The device of claim 34 wherein the compound is selected from the group consisting of penicillins, tetracyclines, sodium fluoride, potassium nitrate, Lidocaine, iodine containing compounds, chlorhexidine, clotrimazole, Nystatin, ibuprofen and salicylates.

40. The device of claim 31 wherein the slow release formulation is comprised of a biodegradable, biocompatible gel.

41. The device of claim 40 wherein the gel comprises hydroxy alkyl methyl cellulose.

42. A dental device for the delivery of a composition to the oral cavity comprised of a crown or dentures, wherein said composition is incorporated in said crown or dentures in a manner that allows slow release of the composition.

43. The device of claim 42 wherein the base of the dentures is comprised of plastic, and the composition is mixed in the plastic.

44. The device of claim 42 wherein the composition is incorporated in the crown or one or more denture teeth.

45. The device of claim 42 wherein the crown is glazed and the composition is incorporated in the glaze.

46. The device of claim 42 wherein the compound is selected from the group consisting of flavorings, anti-bacterials, odor reducers, chemotherapeutics, radiotherapeutics, compositions to improve oral health, anesthetics, antiseptics, antimicrobials, antifungals, anti-inflammatories, antivirals, and combinations thereof.

47. The device of claim 46 wherein the flavorings are flavored oils.

48. The device of claim 46 wherein the odor reducers are selected from the group consisting of chlorine dioxide, zinc gluconate, other biocompatible zinc salts, chlorohexidine, glutaraldehyde, scents and combinations thereof.

49. The device of claim 46 wherein the chemotherapeutics are doxorubicin, vincristine, or a combination thereof.

50. The device of claim 46 wherein the radiotherapeutics are selected from the group consisting of radioactive seeds of ¹²⁵I, ¹⁹²I, palladium, iridium and combinations thereof.

51. The device of claim 46 wherein the compound is selected from the group consisting of penicillins, tetracyclines, sodium fluoride, potassium nitrate, Lidocaine, iodine containing compounds, chlorhexidine, clotrimazole, Nystatin, ibuprofen and salicylates.
